(19) United States
(12) Patent Application Publication
(10) Pub. No.: US 2022/0287924 A1
(43) Pub. Date: Sep. 15, 2022

(54) EMBOSSED DISPERSIBLE WET WIPES

(71) Applicant: Kimberly-Clark Worldwide, Inc., Neenah, WI (US)

(72) Inventors: Robert Stanley Monson, Appleton, WI (US); Nicholas Scott Wolter, Greenville, WI (US); Vickie Marie Thomack, Menasha, WI (US); Daniel Mark Piette, Neenah, WI (US)

(21) Appl. No.: 17/607,920

(22) PCT Filed: Apr. 30, 2019
(86) PCT No.: PCT/US19/29811

§ 371 Date: Nov. 1, 2021

(51) Int. Cl. A61K 8/02; B31F 1/07

(52) U.S. Cl. CPC A61K 8/0208; B31F 1/07; B31F 2201/0735; B31F 2201/0754; B31F 2201/0761; A61K 2800/805

(57) ABSTRACT

The dispersible wet wipes of the current disclosure have sufficient strength to withstand packaging and consumer use. They also disperse sufficiently quickly to be flushable without creating potential problems for household and municipal sanitation systems. In certain instances the wipes have a first outer layer comprising a tissue web containing cellulose fibers and a plurality of embossments disposed thereon, and a second outer layer comprising a nonwoven web; a triggerable binder composition; and a wetting composition. The wipes may have a geometric mean tensile strength (GMT) greater than about 225 grams per linear inch (g/in) and a Slosh Time less than about 60 minutes.

### BACKGROUND

Dispersible moist wipes are generally intended to be used and then flushed down a toilet. Accordingly, it is desirable for such flushable moist wipes to have an in-use strength sufficient to withstand a user's extraction of the wipe from a dispenser and the user's wiping activity, but then relatively quickly breakdown and disperse in household and municipal sanitization systems, such as sewer or septic systems. Some municipalities may define “flushable” through various regulations. Flushable moist wipes must meet these regulations to allow for compatibility with home plumbing fixtures and drain lines, as well as the disposal of the product in onsite and municipal wastewater treatment systems.

One challenge for some known flushable moist wipes is that it takes a relatively longer time for them to break down in a sanitation system as compared to conventional, dry toilet tissue thereby creating a risk of blockage in toilets, drainage pipes, and water conveyance and treatment systems. Dry toilet tissue typically exhibits lower post-use strength upon exposure to tap water, whereas some known flushable moist wipes require a relatively long period of time and/or significant agitation within tap water for their post-use strength to decrease sufficiently to allow them to disperse. Attempts to address this issue, such as making the wipes to disperse more quickly, may reduce the in-use strength of the flushable moist wipes below a minimum level deemed acceptable by users.

Thus, there is a need to provide a wet wipe that provides an in-use strength expected by consumers, disperses sufficiently quickly to be flushable without creating potential problems for household and municipal sanitation systems, and is cost-effective to produce.

### SUMMARY

The present invention provides dispersible wet wipes having improved in-use tensile strength, such as geometric mean tensile (GMT) strengths greater than about 225 grams per linear inch (g/in), more preferably greater than about 250 g/in, such as from about 225 to about 500 g/in, yet the ability of such wipes to disperse in a timely fashion is correspondingly reduced. For example, the wipes may have a Slosh Time less than about 60 minutes, such as from about 20 to about 60 minutes, and more preferably from about 20 to about 40 minutes. Thus, the present invention provides a wet wipe that provides an in-use strength expected by consumers, disperses sufficiently quickly to be flushable without creating potential problems for household and municipal sanitation systems, and is cost-effective to produce.

Generally the improvement in in-use strength and dispersability is achieved by providing an embossed substrate and treating the substrate with a triggerable binder. More particularly, the substrate may comprise a plurality of embossments, such as discrete embossments, that are disposed in a pattern where the pattern covers from about 5 to about 15 percentage of the surface area of the wipe. In a particularly preferred embodiment the embossments are line elements and more preferably curvilinear line elements having a width from about 0.5 to about 2.5 mm, such as from about 0.75 to about 2.0 mm, such as from about 1.0 to about 1.5 mm.

Accordingly, in one embodiment the present invention provides a wipe comprising a wipe substrate having at least a first outer layer comprising a tissue web containing cellulose fibers and a background pattern with a plurality of embossments disposed thereon, and a second outer layer comprising a nonwoven web; a triggerable binder composition; and a wetting composition, wherein the dispersible wet wipe has a geometric mean tensile strength (GMT) greater than about 225 g/in and a Slosh Time less than about 60 minutes. Preferably the background pattern is not embossed and the embossed area is less than about 15 percent and more preferably less than about 10 percent.

In another embodiment the present invention provides a wipe comprising a wipe substrate having at least a first outer layer comprising a tissue web containing cellulose fibers and having a background pattern consisting essentially of a plurality of spaced apart, parallel, continuous curvilinear line elements and a plurality of embossments overlaying the background pattern and arranged in a pattern substantially oriented in the MD and covering from about 5 to about 15 percentage of the surface area of the first outer layer, and a second outer layer comprising a nonwoven web; a triggerable binder composition; and a wetting composition, wherein the dispersible wet wipe has a geometric mean tensile strength (GMT) greater than about 225 g/n and a Slosh Time less than about 60 minutes.

In still other embodiments the present invention provides a dispersible wipe having a first outer layer comprising a cellulosic tissue web having a background pattern and a plurality of embossments disposed in a pattern, wherein the background pattern and the embossing pattern are visually related to one another. For example, the background pattern and the embossing pattern may both comprise curvilinear line elements having a maximum line width from about 0.5 to about 2.5 mm. In a particularly preferred embodiment the line elements forming the background pattern are substantially machine direction oriented and the elements forming the embossing pattern are substantially cross-machine direction oriented. Generally the foregoing wipes have improved in-use strength, such as a GMT from about 250 to about 500 g/n, yet disperse readily, such as a Slosh Time from about 20 to about 60 minutes.

In other embodiments the present invention provides a method of forming a dispersible wet wipe comprising the steps of: forming a first fibrous layer comprising a cellulosic tissue web; forming a second fibrous layer comprising a nonwoven web; combining the first and the second layers to form a multi-layered web; conveying the multi-layered web through an embossing nip; embossing the multi-layered web thereby forming a plurality of embossments on at least the first fibrous layer of the multi-layered web; and applying a triggerable binder composition to at least the first or the second fibrous layer of the multi-layered web.

In other embodiments the wipe of the present invention is embossed by conveying a web comprising a wet laid tissue layer and a nonwoven layer through an extended embossing nip formed between an embossing roll having a circumference and an embossing pattern disposed upon a surface thereof; and a continuous belt supported by at least two rolls juxtaposed in an axially parallel relationship with the embossing roll; wherein the continuous belt is disposed upon the circumference of the embossing roll from about 90 degrees of said circumference to about 180 degrees of the circumference. In particularly preferred embodiments the extended nip has nip pressure greater than about 100 pounds per linear inch (pli), such as from about 100 to about 150 pli.

### DESCRIPTION OF THE DRAWINGS

FIG. 1 is a cross-sectional view of the dispersible wet wipe disclosed herein.

FIG. 2 is a schematic illustration of an uncreped through-air dried tissue making process to form an exemplary first layer of the dispersible wet wipe.

FIG. 3 is a schematic illustration of an air laying forming apparatus to form an exemplary second layer of the dispersible wet wipe.

FIG. 4A is a schematic illustration of an exemplary process to form the wipe substrate.

FIGS. 4B-4D are enlargements of certain points in the process of FIG. 4A.

FIG. 5 is an embossing pattern useful in the present invention.

FIG. 6 is another embossing pattern useful in the present invention.

FIG. 7 is a top plane view of the embossed dispersible wipe disclosed herein.

FIG. 8 is a schematic illustration of an embossing and binding process to form a wipe substrate.

FIG. 9 is a photograph of an embossing roll used to manufacture and exemplary wipe substrate.

FIG. 10 is a graph plotting geometric mean tensile strength (g/in) versus Slosh Time (min.) for control and inventive wipes.

### DEFINITIONS

As used herein the term “Machine Direction” or “MD” generally refers to the direction in which a tissue web or product is produced. The term “Cross-Machine Direction” or “CD” refers to the direction perpendicular to the machine direction.

As used herein the term “Embossed” generally refers to a dispersible wipe that has been subjected to a process which passes one or more plies of the wipe through a nip created by one or more embossed rolls having an embossing pattern disposed thereon. Embossed does not include creping, microcreping, printing or other processes that may impart a texture and/or decorative pattern to a fibrous structure.

As used herein the term “Line embossment” generally refers to an embossment that comprises a line element aspect ratio of greater than about 2:1, more preferably greater than about 5:1 and still more preferably greater than about 10:1.

As used herein the term “Line Element” refers to an element in the shape of a line, which may be continuous, discrete, interrupted, or a partial line with respect to dispersible wipe on which it is present. The line element may be of any suitable shape such as straight, curled, curvilinear, and mixtures thereof. In one example, the line element may comprise a plurality of discrete elements, such as dots, dashes or broken lines for example, that are oriented relative to one another to form a line element having a substantially connected visual appearance.

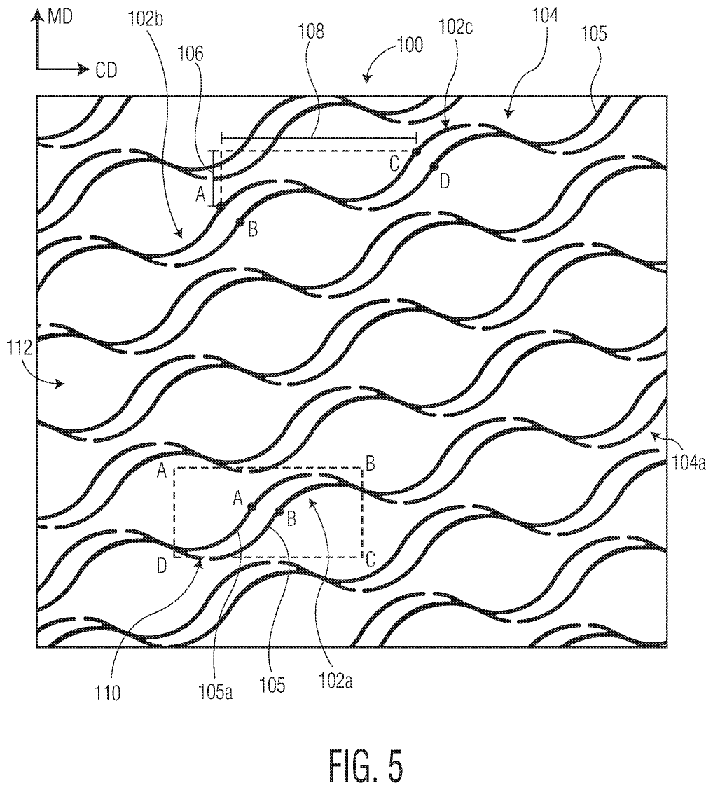

As used herein the term “Continuous” when referring to an element or pattern disposed on the surface of a dispersible wipe means that the element or pattern extends throughout one dimension of the dispersible wipe surface. A non-limiting example of a continuous pattern is illustrated in FIG. 5 where the emboss pattern 100 is a continuous pattern in the form of repeating, connected, S-shaped elements 102 that form a motif 104. The emboss pattern 100 is continuous despite individual S-shaped elements 102 having a break 110.

As used herein the term “Discrete” when referring to an element or pattern disposed on the surface of a dispersible wipe means that the element or pattern is visually unconnected from other elements and does not extend continuously in any dimension of the dispersible wipe surface.

As used herein the term “Curvilinear Element” refers to any curved line element having at least one inflection point. A curvilinear line element need not be a continuous line, but rather may comprise discrete dots, dashes or line segments that are substantially connected visually. For example, with reference to FIG. 5, the motif 104 comprises a plurality of curvilinear elements 102 that are generally S-shaped. Despite the line breaks 110 the curvilinear element 102 has the appearance of being substantially connected. Curvilinear elements may be used to form one or more elements according to the present invention. In certain embodiments an element may be formed from a single curvilinear line element or by a pair of spaced apart line elements.

As used herein the term “MD Segment Length” generally refers to the distance in the machine direction between two adjacent inflection points within a single curvilinear line element having two or more inflection points. Where a curvilinear line element has only a single inflection point, the MD segment length is measured in the machine direction between the inflection points of adjacent curvilinear elements within a motif where the motif comprises more than one curvilinear element or between the inflection points of curvilinear elements in adjacent motifs where the motif comprises only a single curvilinear element.

As used herein the term “CD Segment Length” generally refers to the distance in the cross-machine direction between two adjacent inflection points within a single curvilinear element having two or more inflection points. Where a curvilinear line element has only a single inflection point, the CD segment length is measured in the cross-machine direction between the inflection points of adjacent curvilinear elements within a motif where the motif comprises more than one curvilinear element or between the inflection points of curvilinear elements in adjacent motifs where the motif comprises only a single curvilinear element.

As used herein the term “Pattern” generally refers to the arrangement of one or more elements. Within a given pattern the elements may be the same or may be different, further the elements may be the same relative size or may be different sizes. For example, in one embodiment, a single element may be repeated in a pattern, but the size of the design element may be different from one element to the next within the pattern.

As used herein, the term “Embossing Pattern” generally refers to the arrangement of one or more design elements across at least one dimension of a dispersible wipe surface that are imparted by embossing the dispersible wipe. The pattern may comprise a linear element, a non-linear element, a discrete non-linear element or other shapes. The embossing pattern comprises a portion of the dispersible wipe lying out of plane with the surface plane of the dispersible wipe. In general, the embossing pattern results from embossing the dispersible wipe resulting in a depressed area having a z-directional elevation that is lower than the surface plane of the dispersible wipe. The depressed areas can suitably be one or more linear elements, discrete elements or other shapes.

As used herein, the term “Embossment Plane” generally refers to the plane formed by the upper surface of the depressed portion of the dispersible wipe forming an embossment. Generally the embossing element plane lies below the dispersible wipe's surface plane. In certain embodiments the dispersible wipe of the present invention may have a single embossing element plane, while in other embodiments the structure may have multiple embossing element planes. The embossing element plane is generally determined by imaging a cross-section of the dispersible wipe and drawing a line tangent to the upper most surface of an embossment where the line is generally parallel to the x-axis of the dispersible wipe.

As used herein the term “Embossed Area” generally refers to the percentage of a dispersible wipe's surface area that is covered by embossments as measured using a Keyence VHX-5000 Digital Microscope (Keyence Corporation, Osaka, Japan) and described in the Test Methods section below.

As used herein the term “Background Pattern” refers to a pattern that substantially covers the surface of a dispersible wipe. One of skill in the art may appreciate that a background pattern may be distinguished from a repeating pattern because a repeating pattern may comprise a plurality of line segment patterns, line segment axes, and cells whereas, in some embodiments, a background pattern may only comprise a single feature which is repeated at any frequency and/or interval. In other embodiments, a background pattern comprises a plurality of features which may form a repeating unit. A repeating unit may be described as a design comprising a plurality of one or more base patterns.

A background pattern may be formed using any means known in the art. For example, in some embodiments, a background pattern may be introduced into the surface of a dispersible wipe using embossing or micro-embossing. Exemplary embodiments of micro embossing are described, for example, in US Publication No. 2005/0230069. In other embodiments, a background pattern may be introduced into the surface of the tissue sheet or product during the papermaking process using a textured or patterned papermaking fabric as described in, for example, U.S. Pat. No. 7,611,607.

As used herein the term “Motif” generally refers to the recurrence of one or more elements within a pattern. The recurrence of the element may not necessarily occur within a given sheet, for example, in certain embodiments the element may be a continuous element extending across two adjacent sheets separated from one another by a line of perforations. Motifs are generally non-random repeating units that form a pattern.

As used herein, the term “Basis Weight” generally refers to the bone dry weight per unit area of a tissue and is generally expressed as grams per square meter (gsm). Basis weight is measured using TAPPI test method T-220.

As used herein, the term “Ply” refers to a discrete product element. Individual plies may be arranged in juxtaposition to each other. The term may refer to a plurality of web-like components such as in a multi-ply facial tissue, multi-ply bath tissue, multi-ply paper towel, multi-ply wipe, or multi-ply napkin, which may comprise two, three, four or more individual plies arranged in juxtaposition to each other where one or more plies may be attached to one another such as by mechanical or chemical means.

As used herein, the term “Layer” refers to a plurality of strata of fibers, chemical treatments, or the like, within a ply. In certain instances layers within a given ply may be manufactured by different manufacturing techniques. For example a ply may comprise a first layer formed by wet laying fibers and a second layer formed by air laying fibers.

As used herein, the term “Caliper” is the representative thickness of a single sheet (caliper of dispersible wipes comprising one or more plies is the thickness of a single sheet of dispersible wipe comprising all plies) measured in accordance with TAPPI test method T402 using a ProGage 500 Thickness Tester (Thwing-Albert Instrument Company, West Berlin, N.J.). The micrometer has an anvil diameter of 2.22 inches (56.4 mm) and an anvil pressure of 132 grams per square inch (per 6.45 square centimeters) (2.0 kPa).

As used herein, the term “Geometric Mean Tensile” (GMT) refers to the square root of the product of the machine direction tensile strength and the cross-machine direction tensile strength of the web. While the GMT may vary, dispersible wipes prepared according to the present disclosure may, in certain embodiments, wipes prepared according to the present invention have a GMT greater than about 225 g/n, more preferably greater than about 250 g/n, and more preferably greater than about 275 g/n and still more preferably greater than about 300 g/n, such as from about 225 to about 500 g/n, such as from about 250 to about 400 g/n.

### DESCRIPTION

The dispersible wet wipes of the current disclosure have sufficient strength to withstand packaging and consumer use. They also disperse sufficiently quickly to be flushable without creating potential problems for household and municipal sanitation systems. Additionally, they may be comprised of materials that are suitably cost-effective.

The present disclosure is thus directed to, in part, an embossed dispersible wet wipe constructed of at least two layers having a binder, such as a triggerable binder composition that binds the layers together. Generally the layers have different densities and in certain instances may be made by different manufacturing processes and may comprise different fiber furnishes. For example, the first outer layer of the wipe substrate may have a density of between about 0.5 and 2.0 grams per cubic centimeter and the second outer layer may have a density of between about 0.05 and 0.15 grams per cubic centimeter. By providing an embossed wipe having different layers bound together by a triggerable binder, the wipe demonstrates high initial wet strength and rapid loss in wet strength under static soak. This combination has the surprising effect of a high initial strength and effective dispersion and can be used as, for example, a flushable surface cleaning product or a flushable cleansing cloth.

In accordance with the present disclosure, the inventors have surprisingly found a solution for a dispersible wipe with greater wet strength than conventional wipes and improved dispensability by embossing the wipe substrate after application of a binder, but prior to curing of the binder by drying the substrate. Thus, in one embodiment of the present disclosure, a method for making a dispersible wipe is disclosed, the method comprising the steps of: forming a first fibrous layer comprising a cellulosic tissue web; forming a second fibrous layer comprising a nonwoven web; combining the first and the second layers to form a multi-layered web; conveying the multi-layered web through an embossing nip; embossing the multi-layered web thereby forming a plurality of embossments on at least the first fibrous layer of the multi-layered web; and applying a triggerable binder composition to at least the first or the second fibrous layer of the multi-layered web. By embossing the binder treated substrate before curing of the binder the inventors were able to increase the strength of wet wipes while still maintaining a good dispersibility.

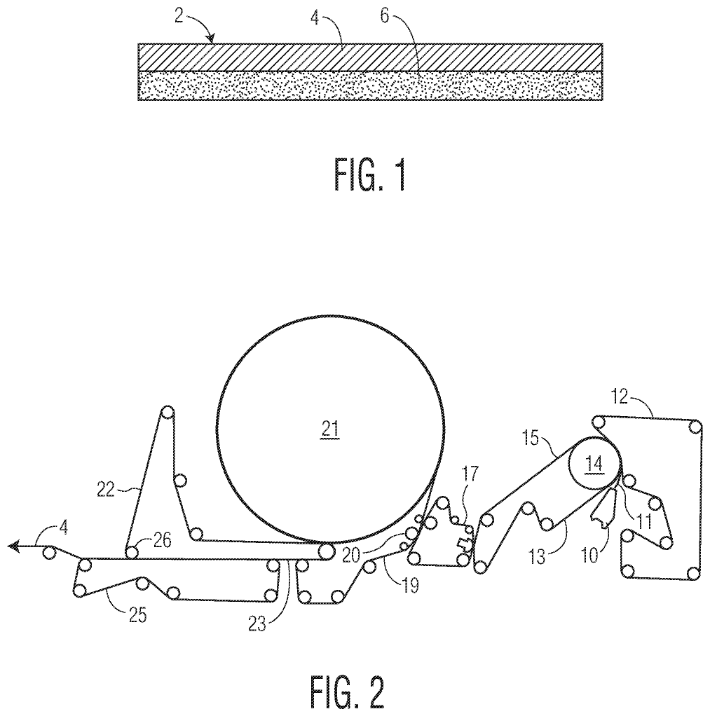

Referring to FIG. 1, a dispersible wet wipe is illustrated having as least two outer layers. The first layer of the wipe substrate may have a density of between about 0.5 and 2.0 grams per cubic centimeter. Typically, the first layer of the fibrous substrate may have a basis weight of from about 20 to about 100 grams per square meter and desirably from about 20 to about 90 grams per square meter. Most desirably, the wipes of the present disclosure define a basis weight from about 30 to about 75 grams per square meter.

Materials suitable for the substrate of the wipes are well known to those skilled in the art, and are typically made from a fibrous sheet material which may be either woven or nonwoven. Two types of nonwoven materials are described herein, the “nonwoven fabrics” and the “nonwoven webs”. The nonwoven material may comprise either a nonwoven fabric or a nonwoven web. The nonwoven fabric may comprise a fibrous material, while the nonwoven web may comprise the fibrous material and a binder composition. In another embodiment, as used herein, the nonwoven fabric comprises a fibrous material or substrate, where the fibrous material or substrate comprises a sheet that has a structure of individual fibers or filaments randomly arranged in a mat-like fashion, and does not include the binder composition. Since nonwoven fabrics do not include a binder composition, the fibrous substrate used for forming the nonwoven fabric may desirably have a greater degree of cohesiveness and/or tensile strength than the fibrous substrate that is used for forming the nonwoven web. For this reason nonwoven fabrics comprising fibrous substrates created via hydroentangling may be particularly preferred for formation of the nonwoven fabric. Hydroentangled fibrous materials may provide the desired in-use strength properties for wet wipes that comprise a nonwoven fabric.

For example, suitable materials for use in the wipes may include nonwoven fibrous sheet materials which include tissue, meltblown, coform, airlaid, bonded-carded web materials, hydroentangled materials, spunlace materials, and combinations thereof. Such materials can be comprised of synthetic or natural fibers, or a combination thereof.

Desirably, the first layer of the dispersible wipes is constructed from tissue webs. Basesheets suitable for this purpose can be made using any process that produces a high density, resilient tissue structure. Such processes include uncreped through-air dried, creped through-air dried and modified wet press processes. Desirably, the first layer of the wipe substrate is an uncreped through-air dried tissue basesheet. Exemplary processes to prepare uncreped through-air dried tissue are described in U.S. Pat. Nos. 5,607,551, 5,672,248, 5,593,545, 6,083,346 and 7,056,572, all herein incorporated by reference.

FIG. 2 illustrates a machine for carrying out the method of forming the first layer of the wipe defined herein. (For simplicity, the various tensioning rolls schematically used to define the several fabric runs are shown but not numbered. It will be appreciated that variations from the apparatus and method illustrated in FIG. 2 can be made without departing from the scope of the claims.) Shown is a twin wire former having a layered papermaking headbox 10 which injects or deposits a stream 11 of an aqueous suspension of papermaking fibers onto the forming fabric 13 which serves to support and carry the newly-formed wet web downstream in the process as the web is partially dewatered to a consistency of about 10 dry weight percent. Additional dewatering of the wet web can be carried out; such as by vacuum suction, while the wet web is supported by the forming fabric.

The wet web is then transferred from the forming fabric to a transfer fabric 17 traveling at a slower speed than the forming fabric in order to impart increased stretch into the web. Transfer is preferably carried out with the assistance of a vacuum shoe 18 and a fixed gap or space between the forming fabric and the transfer fabric or a kiss transfer to avoid compression of the wet web.

The web is then transferred from the transfer fabric to the through-air drying fabric 19 with the aid of a vacuum transfer roll 20 or a vacuum transfer shoe, optionally again using a fixed gap transfer as previously described. The through-air drying fabric can be traveling at about the same speed or a different speed relative to the transfer fabric. If desired, the through-air drying fabric can be run at a slower speed to further enhance stretch. Transfer is preferably carried out with vacuum assistance to ensure deformation of the sheet to conform to the through-air drying fabric, thus yielding desired bulk and appearance.

The level of vacuum used for the web transfers can be from about 3 to about 15 inches of mercury (75 to about 380 millimeters of mercury), preferably about 5 inches (125 millimeters) of mercury.

The vacuum shoe (negative pressure) can be supplemented or replaced by the use of positive pressure from the opposite side of the web to blow the web onto the next fabric in addition to or as a replacement for sucking it onto the next fabric with vacuum. Also, a vacuum roll or rolls can be used to replace the vacuum shoe(s).

While supported by the through-air drying fabric, the web is final dried to a consistency of about 94 percent or greater by the through-air dryer 21 and thereafter transferred to a carrier fabric 22. The dried basesheet 23 that is prepared is the first layer of the dispersible wipe. An optional pressurized turning roll 26 can be used to facilitate transfer of the web from carrier fabric 22 to fabric 25. Suitable carrier fabrics for this purpose are Albany International 84M or 94M and Asten 959 or 937, all of which are relatively smooth fabrics having a fine pattern. Although not shown, reel calendering or subsequent off-line calendering can be used to improve the smoothness and softness of the first layer of the basesheet. The resulting sheet produced is the first layer of the dispersible substrate.

Desirably, the first layer comprises fibers having a length weighted average fiber length less than about 3.0 mm, such as from about 1.0 to about 3.0 mm and more preferably wood pulp fibers having a length weighted average fiber length less than about 3.0 mm. For example, in certain instances the first layer may consist essentially of wood pulp fibers having a length weighted average fiber length from about 1.0 to about 3.0 mm such as, for example, a blend of hardwood and softwood kraft pulp fibers.

Referring again to FIG. 1, the second outer layer of the wipe substrate may have a density of between about 0.05 and 0.15 grams per cubic centimeter. Typically, the first layer of the fibrous substrate may have a basis weight of from about 10 to about 100 grams per square meter and desirably from about 10 to about 60 grams per square meter. Most desirably, the wipes of the present disclosure define a basis weight from about 10 to about 45 grams per square meter. The two substrates are embossed together to bring the fibers closer together, ensuring proper bonding of the two outer layers.

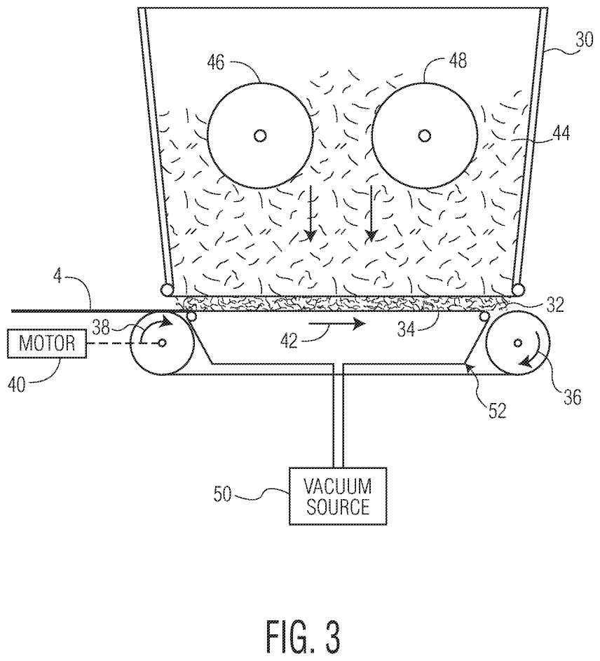

One embodiment of a process for forming the second layer as described herein will now be described in detail with particular reference to FIG. 3. It should be understood that the air laying apparatus illustrated in FIG. 3 is provided for exemplary purposes only and that any suitable air laying equipment may be used in the process.

Various suitable forming fabrics for use can be made from woven synthetic strands or yarns. One suitable forming fabric is an ElectroTech 100S, available from Albany International having an office in Albany, N.Y. The ElectroTech 100S fabric is a 97 by 84 count fabric with an approximate air permeability of 575 cfm, an approximate caliper of 0.048 inch, and a percent open area of approximately 0 percent.

As shown, the air laying forming station 30 includes a forming chamber 44 having end walls and side walls. Within the forming chamber 44 are a pair of material distributors which distribute fibers and/or other particles inside the forming chamber 44 across the width of the chamber. The material distributors can be, for instance, rotating cylindrical distributing screens.

In the embodiment shown in FIG. 3, a single forming chamber 44 is illustrated in association with the forming fabric 34. It is understood that more than one forming chamber can be included in the system. By including multiple forming chambers, layered webs can be formed in which each layer is made from the same or different materials.

Air laying forming stations, as shown in FIG. 3, are available commercially through Dan-Webforming International LTD. of Aarhus, Denmark. Other suitable air laying forming systems are also available from Oerlikon-Neumag of Horsens, Denmark. As described above, any suitable air laying forming system can be used to prepare the second layer of the wipe substrate described herein.

As shown in FIG. 3, below the air laying forming station 30 is a vacuum source 50, such as a conventional blower, for creating a selected pressure differential through the forming chamber 44 to draw the fibrous material against the first layer 4 residing on the forming fabric 34. If desired, a blower can also be incorporated into the forming chamber 44 for assisting in blowing the fibers down onto the forming fabric 34.

In one embodiment, the vacuum source 50 is a blower connected to a vacuum box 52, which is located below the forming chamber 44 and the forming fabric 34. The vacuum source 50 creates an airflow indicated by the arrows positioned within the forming chamber 44. Various seals can be used to increase the positive air pressure between the chamber and the forming fabric surface.

During operation, typically a fiber stock is fed to one or more defibrators (not shown) and fed to the material distributors. The material distributors distribute the fibers evenly throughout the forming chamber 44 as shown. Positive airflow created by the vacuum source 50, and possibly an additional blower, forces the fibers onto the first layer 4, thereby forming an air laid nonwoven web 32.

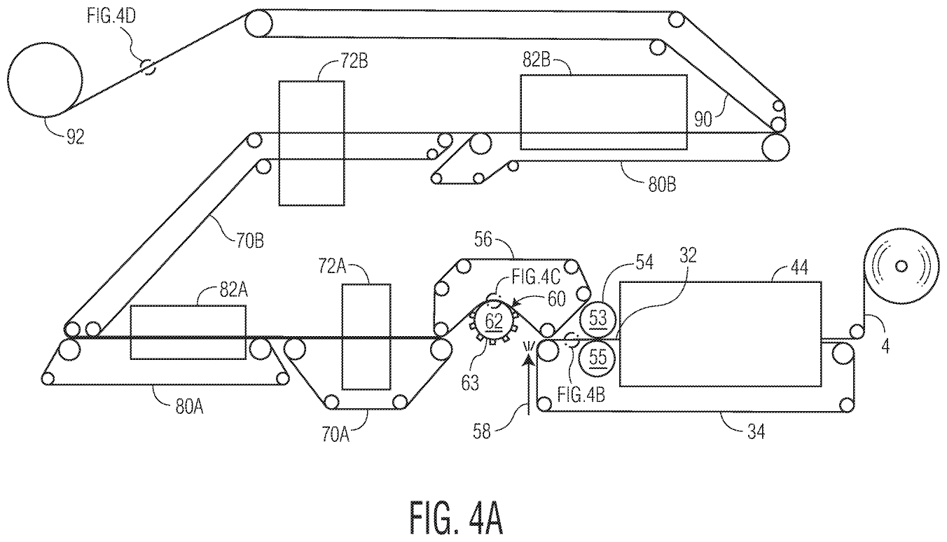

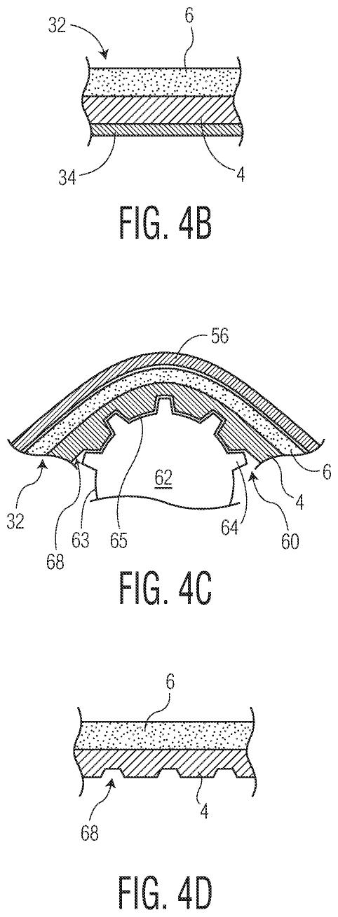

In FIG. 4A, a schematic diagram of an entire web forming system useful for making multi-layered substrates is shown. In this embodiment, the system includes an air laying forming chamber 44. As described above, the use of multiple forming chambers can serve to facilitate formation of the air laid web at a desired basis weight. Further, using multiple forming chambers can allow for the formation of layered webs. As shown, forming station 44 contributes to the formation of the dual layer substrate.

The first layer 4, which is preferably a tissue base sheet, is unwound onto a forming fabric 34 and conveyed through the forming chamber 44 having end walls and side walls. Within the forming chamber 44 fibers and/or other particles are distributed across the width of the chamber onto the first layer 4 while it is supported by the forming fabric 34 to a form a two-layered web 32.

The two-layered web 32 (shown in detail in FIG. 4B), after exiting the forming chambers 44, is conveyed to a compaction device 54. The compaction device 54 can be a pair of opposing rolls that define a nip through which the air laid web and forming fabric is passed. In one embodiment, the compaction device can comprise a steel roll 53 positioned above a covered roll 55, having a resilient roll covering for its outer surface.

The compaction rolls 53, 55 can be between about 10 to about 30 inches in diameter and can be optionally heated to further enhance their operation. For example, the compaction rolls 53, 55 may comprise a pair of steel rolls, which in certain instances may be heated to a temperature from about 60 to about 200° C. The compaction rolls can be operated at either a specified loading force or can be operated at a specified gap between the surfaces of each roll. Too much compaction will cause the web to lose bulk in the finished product, while too little compaction can cause runnability problems when transferring the air laid web to the next section in the process.

Alternatively, the compaction device 54 can be eliminated and the transfer fabric 56 and the forming fabric 34 can be brought together such that the two-layered web 32 is transferred from the forming fabric to the transfer fabric 56. The transfer efficiency can be enhanced by use of suitable vacuum transfer boxes and/or pressured blow boxes as known in the art.

Regardless of whether or not the two-layered web is compacted by a pair of compaction rolls, it may be desirable to hydrate the web before further processing. For example, as shown in FIG. 4A a liquid, such as water, may be applied to one of the outer surfaces two-layered web 32 by a spray boom 58. The percent moisture of the air laid web after hydration, based as a weight percent of the dry fibers of the web, can be between about 0.1 to about 5 percent, or between about 0.5 to about 4 percent, or between about 0.5 to about 2 percent. Too much moisture can cause the air laid web to adhere to the transfer fabric and not release for transfer to the next section of the process, while too little moisture can reduce the amount of texture generated in the web.

Next, while supported by the transfer fabric 56, the two-layered web 32 is embossed by passing the web 32 through a nip 60 created between the transfer fabric 56 and an embossing roll 62. As shown in detail in FIG. 4C, the transfer fabric 56 may be positioned adjacent to the embossing roll 62 such that the roll surface 63 and the surface of the transfer fabric 56 are in contact or overlap one another to form an extended emboss nip 60.

The distance and time the web 32 is in contact with the embossing roll 62 may be controlled by the contact or overlap between the transfer fabric 56 and the roll surface 63. For example, the portion of the roll surface 63 contacting the transfer fabric 56 may range from 2 degrees of the embossing roll circumference to as much as 200 degrees of the embossing roll circumference. To control the degree of contact the transfer fabric 56 may be maintained in a fixed position and the embossing roll 62 can be adjusted relative to the transfer fabric 56. In any regard, it is preferable that the transfer fabric 56 be loaded against the embossing roll 62 in order to achieve the desired embossment. Further, in certain instances, in addition to controlling the degree of contact between the roll surface 63 and transfer fabric 56, the relative speed of the two may be controlled such that they substantially similar surface speeds.

In particularly preferred embodiments embossing is performed using an embossing apparatus having an extended nip, such as that disclosed in US Publication No. 2010/00294450, the contents of which are incorporated herein in a manner consistent with the present disclosure. In certain preferred embodiments the embossing apparatus may include positioning device (not shown), such as linear actuators, servo motors, cams, links, and the like to control of the position of the belt relative to embossing roll. In this manner the desired contact, clearance, and/or pressure between the belt and the embossing roll may be controlled to provide embossments upon the web, particularly the tissue layer of web, as desired.

The transfer fabric 56 is preferably made of flexible material so as to conform to the embossing roll 62, but should also be sufficiently rigid to hold the form of the embossing pattern. Examples of fabric material include but are not limited to rubber, polyurethane, nylon, polyesters, and polytetrafluoroethylene. The transfer fabric 56 may comprise a deformable surface such as a synthetic rubber as known in the art which, when loaded against the embossing roll 62 with web 32 disposed on the embossing roll surface 63 (which consists of protuberances 64 and land areas 65), deforms the web 32 on and around the protuberances 64 thereby imparting the desired embossment 68 onto the web 32.

In certain preferred embodiments the transfer fabric may be provided with a relieved surface or complimentary to the embossing pattern disposed upon the embossing roll. In this embodiment, the relief portions can be provided as a pattern disposed upon or within the material comprising transfer fabric. The transfer fabric position may be controlled such that the distal ends of the transfer fabric pattern elements extend into any relieved portion corresponding to any protuberances disposed upon the embossing roll. The depth of engagement between the transfer fabric pattern elements and the protuberances disposed upon embossing roll, as well as any clearance between mating pattern elements, can be controlled to impart a desired embossing image onto the web.

The embossing roll 62, which is generally a steel roll includes a plurality of discrete male embossing elements, also referred to herein as protuberances 64, alternatively referred to as male embossing elements, are arranged in a embossing pattern and separated from one another by relatively smooth land areas 65. The protuberances 64 are raised above the land areas 65 and are pressed into the two-layered web to form a corresponding image of the embossing pattern when the web is processed through the embossing nip 60. In particularly preferred embodiments the protuberances 64 contact and compress the first layer 4, which is generally a tissue layer, of the two-layered web 32 to form an embossment 68 (shown in detail in FIG. 4D).

The male embossing elements protrude from the land areas by a distance or height H, of about 0.15 mm or greater, such as from about 00.15 to about 2.0 mm, and preferably from about 0.75 to about 1.02 mm. The width of the male embossing elements at the tip is typically from about 0.5 to about 2.5 mm, such as from about 0.75 to about 2.0 mm, such as from about 1.0 to about 1.5 mm. The sidewall angle, theta (θ), as measured relative to the plane tangent to the surface of the roll at the base of the embossing element, is from about 90 degrees to about 130 degrees. The embossing roll 30 is formed by engraving or other techniques known in the art. Generally the male embossing elements define a decorative pattern, which may include geometric patterns. Particularly desirable patterns will be discussed in more detail below.

In certain embodiments the embossing elements may be heated using traditional methods such as by circulating hot oil or water inside the embossing roll. Heated embossing elements affect the embossing pattern of the web. Generally, the heated elements reduce the web's resiliency thereby creating a more rigid and defined pattern. The extent of heat is dictated by the desired embossing pattern.

In certain instances the embossing apparatus may further comprise a press shoe disposed adjacent the transfer fabric and opposite the embossing roll. The press shoe preferably has a concave surface substantially matching the curvature of the embossing roll. In operation, a force is applied to the press shoe, urging the shoe towards the transfer fabric and conforming the fabric to the embossing roll. The force applied to the press show may be controlled so as to control the nip load and the degree to which the transfer fabric is partially wrap around an arc of the embossing roll. When pressure is applied to the press shoe and the fabric is partially about the engraved roll an extended nip is formed, which generally defines the embossing zone through which the two-layered web is passed and imparted with an embossing pattern.

Next, the two-layered web 32 is transferred to a spray fabric 70A and fed to a spray chamber 72A. Within the spray chamber 72A, a binder is applied to one side of the two-layered web 32. The binder material can be deposited on the top side of the web using, for instance, spray nozzles. Under fabric vacuum may also be used to regulate and control penetration of the binder material into the web.

Once the binder material is applied to one side of the two-layered web 32, as shown in FIG. 4, the two-layered web 32is transferred to drying fabric 80A and fed to a drying apparatus 82A. In the drying apparatus 82A, the web is subjected to heat causing the binder material to dry and/or cure. When using an ethylene vinyl acetate copolymer binder material, the drying apparatus can be heated to a temperature of between about 120 to about 170° C.

From the drying apparatus 82A, the air laid web is then transferred to a second spray fabric 70B and fed to a second spray chamber 72B. In the spray chamber 72B, a second binder material is applied to the other untreated side of the two-layered web 32. The first binder material and the second binder material can be different binder materials or the same binder material. The second binder material may be applied to the air laid web as described above with respect to the first binder material.

From the second spray chamber 72B, the two-layered web 32 is then transferred to a second drying fabric 80B and passed through a second drying apparatus 82B for drying and/or curing the second binder material. From the second drying apparatus 82B, the two-layered web 32 is transferred to a return fabric 90 and then wound into a roll or reel 92. After winding, subsequent converting steps known to those of skill in the art can be used to transform the textured air laid substrate into a plurality of wet wipes. For example, the textured air laid substrate can be cut into individual wipes, the individual the wipes folded into a stack, the stack of wet wipes moistened with a cleaning solution, and then the stack of wet wipes can be placed into a dispenser.

Wipes prepared according to the present invention generally comprise two or more layers and a binder composition, which may be applied to the entire thickness of the wipe, or applied to each individual layer separately and then combined with other layers in a juxtaposed relationship to form the finished wipe. It is also desirable that the wipes of the present invention comprise a plurality of embossments. In certain preferred embodiments the embossments are arranged in a pattern. In certain instances the embossed area may be about 20 percent or less, such as 15 percent or less, such as 12 percent or less, such as from about 5 to about 20 percent or from about 6 to about 12 percent.

With reference now to FIG. 5, one embodiment of an embossing pattern 100 useful in the present invention is illustrated. The embossing pattern 100 comprises a plurality of repeating substantially identical motifs 104. Each motif 104 is made up of interconnected curvilinear elements 102 having an S-shape. With particular reference to curvilinear element 102a, outlined by box ABCD, the element is formed from first and second line elements 105a, 105b each having a single inflection point, noted as points A and B. The curvilinear element 102a has a line break 110, but nonetheless has a continuous visual appearance and when interconnected with other elements forms a motif 104a that extends continuously in the cross-machine direction. Between motifs 104 are pillow regions 112, which like the motifs 104, extend continuously in the CD.

Individual curvilinear elements, such as elements 102b and 102c can be arranged such that the MD and CD segment lengths 106, 108 are both greater than zero. In the illustrated embodiment, the elements 102b and 102c are generally oriented in the CD but are skewed slightly in the MD. The orientation of the second pattern is non-limiting and one skilled in the art will appreciate that the second pattern may be oriented substantially in the CD such that the MD segment length is essentially zero.

Further, while the curvilinear elements 102 forming the embossing pattern 100 are illustrated as having intermittent breaks 110, the invention is not so limited. Rather, the line elements may substantially continuous. Also, while the curvilinear elements 102 are illustrated as being formed from continuous line elements 105, in other embodiments the elements may comprise a plurality discrete dots or dashes that, from a visual perspective, appear to be a continuous line, which may be broken or unbroken. Regardless of whether the element is formed from a broken line element, a continuous line element or a plurality of dots or dashes that are visually connected to give the appearance of a line, it is generally preferred that when viewing an element, the viewer is able to mentally complete the shape so as to perceive a continuous line element.

The size and scale of individual curvilinear elements forming an embossing pattern may vary depending on the desired degree of tensile strength modification, dispensability and aesthetic appearance. In certain instances the pattern may comprise elements arranged and sized such that the MD segment length is from about 10 to about 100 mm, more preferably from about 30 to about 60 mm and still more preferably from about 40 to about 50 mm. In other instances the pattern may comprise elements arranged and sized such that the CD segment length less than, equal to, or greater than the MD segment length, such as from about 10 to about 100 mm, more preferably from about 30 to about 60 mm and still more preferably from about 40 to about 50 mm. Further, the widths of lines forming the curvilinear elements may range from about 0.5 to about 2.5 mm, such as from about 0.75 to about 2.0 mm, such as from about 1.0 to about 1.5 mm.

In one embodiment, at the foregoing spacing and widths, a dispersible wipe of the present invention may have an embossing pattern comprising substantially similarly shaped and sized discrete curvilinear elements that form a continuous motif in at least one dimension of the wipe and wherein the maximum distance between discrete line elements is about 4.0 cm or less, such as from about 1.0 to about 4.0 cm, such as from about 2.0 to about 4.0 cm. In a particularly preferred embodiment the motifs are arranged such that pillow regions are formed there between and like the motifs, extend continuously in at least one dimension of the wipe.

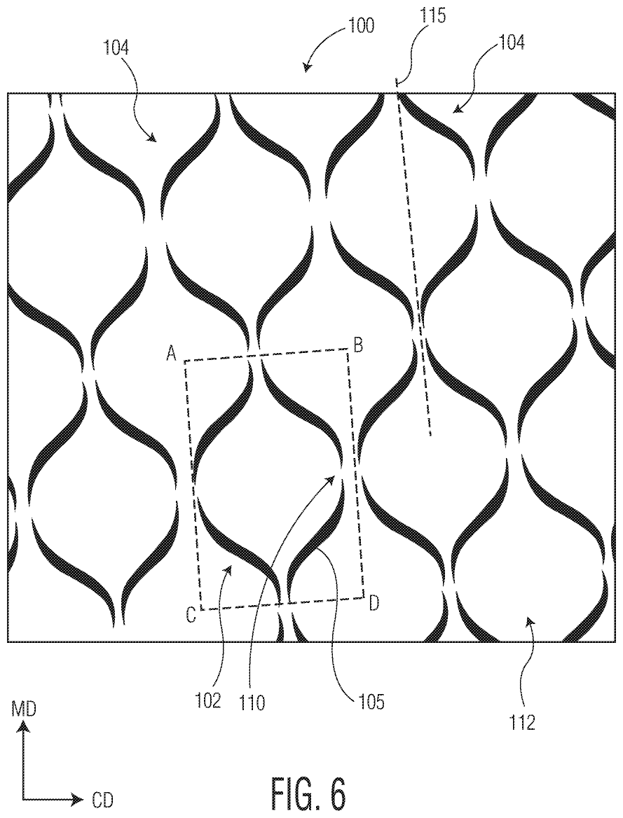

With reference now to FIG. 6, an alternative embossed pattern 100 is illustrated. The pattern 100 comprises a plurality of substantially similarly shaped and oriented motifs 104 that comprise curvilinear elements 102. On element 102 is circumscribed by the box ABCD. The elements 102 are formed from similarly shaped and sized line elements 105 separated from line breaks 110. Despite the presence of line breaks 110 the elements are visually connected to provide a motif that appears to extend continuously in the MD.

Further, in certain instances, the embossed pattern 100 may comprise motifs 104 having an axis of symmetry 115. In this manner the spacing between adjacent motifs may be measured with reference to the adjacent pattern's axis of symmetry. In certain embodiments the dispersible wipe may comprise a pattern having motifs spaced apart from one another continuously throughout the surface of the product where adjacent motifs are spaced apart from one another by at least about 10 mm, such as from about 10 to about 50 mm and more preferably from about 20 to about 30 mm.

In certain preferred embodiments it may be desirable to provide the dispersible wipe with a second pattern, such as a background pattern, that is not embossed but visually relates to the embossed pattern. For example, the wipe, and more particularly a wet-laid layer of the wipe, may comprise a background pattern comprising a curvilinear design element and more particularly a sinusoidal wave having a maximum segment length from about 10 to about 250 mm, more preferably from about 25 to about 100 mm and still more preferably from about 40 to about 60 mm. The embossed pattern overlays the background pattern to provide the wipe with a first and a second pattern.

Preferably the shape of the elements forming the embossed pattern are different than the shape of the elements forming the background pattern. For example, the embossed pattern may comprise S-shaped elements and have only a single point of inflection unlike the background may comprise continuous sinusoidal wave elements having two points of inflection. Regardless of the exact shape of the embossed pattern elements and the background pattern elements, it may be preferred to relate the elements to one another through scale. For example, the elements may have similar segment lengths and line widths.

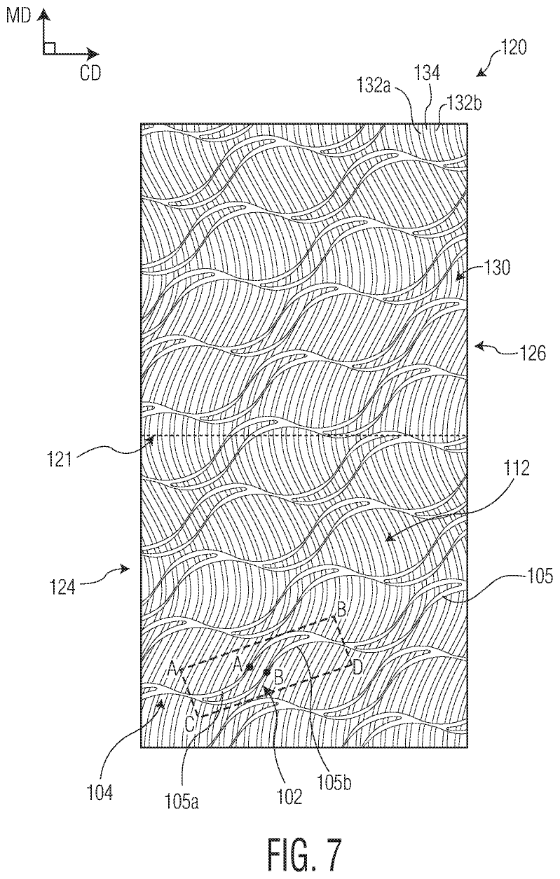

For example, with reference to FIG. 7 the dispersible wipe 120 may have opposed side edges 124, 126 and comprise individual sheets separated by a line of perforations 121. The dispersible wipe 120 has a background pattern 130 comprising a plurality of substantially MD oriented ridges 132a, 132b having a sinusoidal wave shape. The ridges 132a, 132b are separated from one another by valleys 134, which like the ridges 132a, 132b, are imparted to the wipe 120 during manufacture of one of its layers and more particularly are formed by wet molding of a fibrous layer. Overlaying the background pattern 130 is an embossed pattern 100 comprises a plurality of repeating substantially identical motifs 104. Each motif 104 is made up of interconnected curvilinear elements 102 having an S-shape. With particular reference to curvilinear element 102, outlined by box ABCD, the element is formed from first and second line elements 105a, 105b each having a single inflection point, noted as points A and B. The curvilinear element 102 has a line break 110, but nonetheless has a continuous visual appearance and when interconnected with other elements forms a motif 104a that extends continuously in the cross-machine direction. Between motifs 104 are pillow regions 112, which like the motifs 104, extend continuously in the CD.

By providing a wipe having embossed pattern elements and the background pattern elements with similar curvilinear shapes, scale, and line weights the first and second patterns may appear complementary to one another and enhance the overall aesthetic of the dispersible wipe, making it more visually appealing to consumers. Further, by relating the patterns in terms of shape, scale and line weight, the overall design connotations such as femininity, softness and cleansing are enhanced.

Additionally, it may be desirable to form the patterns from curvilinear design elements, which provide for gradual transitions in contour cenote a soothing sensation to consumers. Further, the use of curvilinear design elements enables the formation of open design elements that provide the resulting patterns with a sense of continuity and balance that is visually appealing. A wide breadth of curvilinear design elements may be selected from when developing patterns useful in the present invention. Further, although the patterns of the present invention are formed from curvilinear design elements, one skilled in the art will appreciate that a pattern may include shapes that are not curvilinear as well as lines and other shapes in addition to curvilinear elements.

As described above, the wipe substrate includes a binder composition. In one embodiment the binder composition may include a triggerable polymer. In another embodiment, the binder composition may comprise a triggerable polymer and a co-binder polymer.

The amount of binder composition present in the wipe substrate may desirably range from about 1 to about 15 percent by weight based on the total weight of the wipe substrate. More desirably, the binder composition may comprise from about 1 to about 10 percent by weight based on the total weight of the wipe substrate. Most desirably, the binder composition may comprise from about 3 to about 8 percent by weight based on the total weight of the wipe substrate. The amount of the binder composition results in a multi-ply wipe substrate that has in-use integrity, but quickly disperses when soaked in tap water.

In some embodiments of the present disclosure, the dispersible wipe comprises from about 0.5 grams per square meter (gsm) to about 5 gsm of the binder composition. In preferred embodiments of the present disclosure, the dispersible wipe comprises from about 1 to about 4 gsm, from about 1.2 to about 2.6 gsm, or from about 1.28 to about 2.2 gsm of the binder composition. In other preferred embodiments of the present disclosure, the dispersible wipe comprises about 1.28 gsm, about 1.8 gsm, about 2.2 gsm, about 2.6 gsm, or about 4 gsm of the binder composition.

In one embodiment of the present disclosure, the dispersible wipe comprises triggerable cationic polymer(s) or polymer compositions. The triggerable, cationic polymer composition can be an ion-sensitive cationic polymer composition. In order to be an effective ion-sensitive or triggerable cationic polymer or cationic polymer formulation suitable for use in flushable or water-dispersible personal care products, the formulations should desirably be (1) functional; i.e., maintain wet strength under controlled conditions and dissolve or disperse in a reasonable period of time in soft or hard water, such as found in toilets and sinks around the world; (2) safe (not toxic); and (3) relatively economical. In addition to the foregoing factors, the ion-sensitive or triggerable formulations when used as a binder composition for a non-woven substrate, such as a wet wipe, desirably should be (4) processable on a commercial basis; i.e., may be applied relatively quickly on a large scale basis, such as by spraying (which thereby requires that the binder composition have a relatively low viscosity at high shear); (5) provide acceptable levels of sheet or substrate wettability; (6) provide reduced levels of sheet stiffness; and (7) reduced tackiness. The wetting composition with which the wet wipes of the present disclosure are treated can provide some of the foregoing advantages, and, in addition, can provide one or more of (8) improved skin care, such as reduced skin irritation or other benefits, (9) improved tactile properties, and (10) promote good cleaning by providing a balance in use between friction and lubricity on the skin (skin glide). The ion-sensitive or triggerable cationic polymers and polymer formulations of the present disclosure and articles made therewith, especially wet wipes comprising particular wetting compositions set forth below, can meet many or all of the above criteria.

In some embodiments of the present disclosure, the ion-triggerable cationic polymers of the present disclosure are the polymerization product of a vinyl-functional cationic monomer, and one or more hydrophobic vinyl monomers with alkyl side chain sizes of up to 4 carbons long, such as from 1 to 4 carbon atoms. In preferred embodiments, the ion-triggerable cationic polymers of the present disclosure are the polymerization product of a vinyl-functional cationic monomer, and one or more hydrophobic vinyl monomers with alkyl side chain sizes of up to 4 carbons long incorporated in a random manner. Additionally, a minor amount of another vinyl monomer with linear or branched alkyl groups 4 carbons or longer, alkyl hydroxy, polyoxyalkylene, or other functional groups may be employed.

In one embodiment of the present disclosure, the binder composition comprises a composition having the structure:

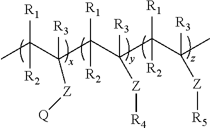

wherein x=1 to about 15 mole percent; y=about 60 to about 99 mole percent; and z=0 to about 30 mole percent; Q is selected from C₁-C₄ alkyl ammonium, quaternary C₁-C₄ alkyl ammonium and benzyl ammonium; Z is selected from —O—, —OOO—, —OOC—, —CONH—, and —NHCO—; R₁, R₂, R_{C1} are independently selected from hydrogen and methyl; R₄ is C₁-C₄ alkyl; R₅ is selected from hydrogen, methyl, ethyl, butyl, ethylhexyl, decyl, dodecyl, hydroxyethyl, hydroxypropyl, polyoxyethylene, and polyoxypropylene.

Vinyl-functional cationic monomers of the present disclosure desirably include, but are not limited to, [2-(acryloxy)ethyl]trimethyl ammonium chloride (ADAMQUAT); [2-(methacryloxy)ethyl)trimethyl ammonium chloride (MADQUAT); (3-acrylamidopropyl)trimethyl ammonium chloride; N,N-diallyldimethyl ammonium chloride; [2-(acryloxy)ethyl]dimethylbenzyl ammonium chloride; (2-(methacryloxy)ethyl]dimethylbenzyl ammonium chloride; [2-(acryloxy)ethyl]dimethyl ammonium chloride; [2-(methacryloxy)ethyl]dimethyl ammonium chloride. Precursor monomers, such as vinylpyridine, dimethylaminoethyl acrylate, and dimethylaminoethyl methacrylate, which can be polymerized and quaternized through post-polymerization reactions are also possible. Monomers or quaternization reagents which provide different counter-ions, such as bromide, iodide, or methyl sulfate are also useful. Other vinyl-functional cationic monomers which may be copolymerized with a hydrophobic vinyl monomer are also useful in the present disclosure.

In some embodiments of the present disclosure, the vinyl-functional cationic monomer is selected from [2-(acryloxy)ethyl]dimethyl ammonium chloride, [2-(acryloxy)ethyl]dimethyl ammonium bromide, [2-(acryloxy)ethyl]dimethyl ammonium iodide, and [2-(acryloxy)ethyl]dimethyl ammonium methyl sulfate.

In some embodiments of the present disclosure, the vinyl-functional cationic monomer is selected from [2-(methacryloxy)ethyl]dimethyl ammonium chloride, [2-(methacryloxy)ethyl]dimethyl ammonium bromide, [2-(methacryloxy)ethyl]dimethyl ammonium iodide, and [2-(methacryloxy)ethyl]dimethyl ammonium methyl sulfate.

In some embodiments of the present disclosure, the vinyl-functional cationic monomer is selected from [2-(acryloxy)ethyl]trimethyl ammonium chloride, [2-(acryloxy)ethyl]trimethyl ammonium bromide, [2-(acryloxy)ethyl]trimethyl ammonium iodide, and [2-(acryloxy)ethyl]trimethyl ammonium methyl sulfate.

In some embodiments of the present disclosure, the vinyl-functional cationic monomer is selected from [2-(methacryloxy)ethyl]trimethyl ammonium chloride, [2-(methacryloxy)ethyl]trimethyl ammonium bromide, [2-(methacryloxy)ethyl]trimethyl ammonium iodide, and [2-(methacryloxy)ethyl]trimethyl ammonium methyl sulfate.

In some embodiments of the present disclosure, the vinyl-functional cationic monomer is selected from (3-acrylamidopropyl)trimethyl ammonium chloride, (3-acrylamidopropyl)trimethyl ammonium bromide, (3-acrylamidopropyl)trimethyl ammonium iodide, and (3-acrylamidopropyl)trimethyl ammonium methyl sulfate.

In some embodiments of the present disclosure, the vinyl-functional cationic monomer is selected from N,N-diallyldimethyl ammonium chloride, N,N-diallyldimethyl ammonium bromide, N,N-diallyldimethyl ammonium iodide, and N,N-diallyldimethyl ammonium methyl sulfate.

In some embodiments of the present disclosure, the vinyl-functional cationic monomer is selected from [2-(acryloxy)ethyl]dimethylbenzyl ammonium chloride, [2-(acryloxy)ethyl]dimethylbenzyl ammonium bromide, [2-(acryloxy)ethyl]dimethylbenzyl ammonium iodide, and [2-(acryloxy)ethyl]dimethylbenzyl ammonium methyl sulfate.

In some embodiments of the present disclosure, the vinyl-functional cationic monomer is selected from [2-(methacryloxy)ethyl]dimethylbenzyl ammonium chloride, [2-(methacryloxy)ethyl]dimethylbenzyl ammonium bromide, [2-(methacryloxy)ethyl]dimethylbenzyl ammonium iodide, and [2-(methacryloxy)ethyl]dimethylbenzyl ammonium methyl sulfate.

Desirable hydrophobic monomers for use in the ion-sensitive cationic polymers of the present disclosure include, but are not limited to, branched or linear C₁-C₁₈ alkyl vinyl ethers, vinyl esters, acrylamides, acrylates, and other monomers that can be copolymerized with the cationic monomer. As used herein the monomer methyl acrylate is considered to be a hydrophobic monomer. Methyl acrylate has a solubility of 6 g/100 ml in water at 20° C.

In some embodiments of the present disclosure, the binder composition comprises the polymerization product of a cationic acrylate or methacrylate and one or more alkyl acrylates or methacrylates having the structure:

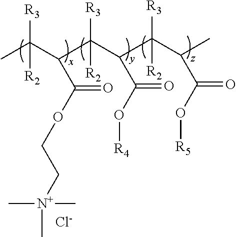

wherein x=1 to about 15 mole percent; y=about 60 to about 99 mole percent; and z=0 to about 30 mole percent; R₄ is C₁-C₄ alkyl; R₅ is selected from hydrogen, methyl, ethyl, butyl, ethylhexyl, decyl, dodecyl, hydroxyethyl, hydroxypropyl, polyoxyethylene, and polyoxypropylene.

In other embodiments of the present disclosure, the binder composition has the structure:

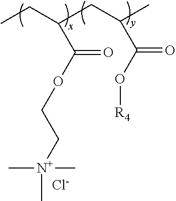

wherein x=1 to about 15 mole percent; y=about 85 to about 99 mole percent and R₄ is C₁-C₄ alkyl. In yet other embodiments of the present disclosure, x=about 3 to about 6 mole percent, y=about 94 to about 97 mole percent and R₄ is methyl. The ion-triggerable cationic polymers of the present disclosure may have an average molecular weight that varies depending on the ultimate use of the polymer. The ion-triggerable cationic polymers of the present disclosure have a weight average molecular weight ranging from about 10,000 to about 5,000,000 grams per mol. More specifically, the ion-triggerable cationic polymers of the present disclosure have a weight average molecular weight ranging from about 25,000 to about 2,000,000 grams per mol, or, more specifically still, from about 200,000 to about 1,000,000 grams per mol.

The ion-triggerable cationic polymers of the present disclosure may be prepared according to a variety of polymerization methods, desirably a solution polymerization method. Suitable solvents for the polymerization method include, but are not limited to, lower alcohols, such as methanol, ethanol and propanol; a mixed solvent of water and one or more lower alcohols mentioned above; and a mixed solvent of water and one or more lower ketones, such as acetone or methyl ethyl ketone.

In the polymerization methods of the present disclosure, any free radical polymerization initiator may be used. Selection of a particular initiator may depend on a number of factors including, but not limited to, the polymerization temperature, the solvent, and the monomers used. Suitable polymerization initiators for use in the present disclosure include, but are not limited to, 2,2′-azobisisobutyronitrile, 2,2′-azobis(2-methylbutyronitrile), 2,2′-azobis(2,4-dimethylvaleronitrile), 2,2′-azobis(2-amidinopropane)dihydrochloride, 2,2′-azobis(N,N′-dimethylene isobutylamidine), potassium persulfate, ammonium persulfate, and aqueous hydrogen peroxide. The amount of polymerization initiator may desirably range from about 0.01 to 5 weight percent based on the total weight of monomer present.

The polymerization temperature may vary depending on the polymerization solvent, monomers, and initiator used, but in general, ranges from about 20 to about 90° C. Polymerization time generally ranges from about 2 to about 8 hours.

In a further embodiment of the present disclosure, the above-described ion-triggerable cationic polymer formulations are used as binder materials for flushable and/or non-flushable products. In order to be effective as a binder material in flushable products throughout the United States, the ion-triggerable cationic polymer formulations of the present disclosure remain stable and maintain their integrity while dry or in relatively high concentrations of monovalent and/or divalent ions, but become soluble in water containing up to about 200 ppm or more divalent ions, especially calcium and magnesium. Desirably, the ion-triggerable cationic polymer formulations of the present disclosure are insoluble in a salt solution containing at least about 0.3 weight percent of one or more inorganic and/or organic salts containing monovalent and/or divalent ions. More desirably, the ion-triggerable cationic polymer formulations of the present disclosure are insoluble in a salt solution containing from about 0.3 to about 10 percent by weight of one or more inorganic and/or organic salts containing monovalent and/or divalent ions. Even more desirably, the ion-triggerable cationic polymer formulations of the present disclosure are insoluble in salt solutions containing from about 0.5 to about 5 percent by weight of one or more inorganic and/or organic salts containing monovalent and/or divalent ions. Especially desirably, the ion-triggerable cationic polymer formulations of the present disclosure are insoluble in salt solutions containing from about 1.0 to about 4.0 percent by weight of one or more inorganic and/or organic salts containing monovalent and/or divalent ions. Suitable monovalent ions include, but are not limited to, Na+ ions, K+ ions, Li+ ions, NH⁴⁺ ions, low molecular weight quaternary ammonium compounds (e.g., those having fewer than 5 carbons on any side group), and a combination thereof. Suitable multivalent ions include, but are not limited to, Zn²⁺, Ca²⁺ and Mg²⁺. The monovalent and divalent ions can be derived from organic and inorganic salts including, but not limited to, NaCl, NaBr, KCl, NH₄Cl, Na₂SO₄, ZnCl₂, CaCl₂, MgCl₂, MgSO₄, NaNO₃, NaSO₄CH₃, and combinations thereof. Typically, alkali metal halides are most desirable because of cost, purity, low toxicity, and availability. A particularly desirable salt is NaCl.

To ensure polymer formulation dispersibility across the country (and throughout the whole world), the ion-triggerable cationic polymer formulations of the present disclosure are desirably soluble in water containing up to about 50 ppm Ca²⁺ and/or Mg²⁺ ions. More desirably, the ion-triggerable cationic polymer formulations of the present disclosure are soluble in water containing up to about 100 ppm Ca²⁺ and/or Mg²⁺ ions. Even more desirably, the ion-triggerable cationic polymer formulations of the present disclosure are soluble in water containing up to about 150 ppm Ca²⁺ and/or Mg²⁺ ions. Even more desirably, the ion-triggerable cationic polymer formulations of the present disclosure are soluble in water containing up to about 200 ppm Ca²⁺ and/or Mg²⁺ ions.

As stated above, the cationic polymer formulations of the present disclosure are formed from a single triggerable cationic polymer or a combination of two or more different polymers, wherein at least one polymer is a triggerable polymer. The second polymer may be a co-binder polymer. A co-binder polymer is of a type and in an amount such that when combined with the triggerable cationic polymer, the co-binder polymer desirably is largely dispersed in the triggerable cationic polymer; i.e., the triggerable cationic polymer is desirably the continuous phase and the co-binder polymer is desirably the discontinuous phase. Desirably, the co-binder polymer can also meet several additional criteria. For example, the co-binder polymer can have a glass transition temperature; i.e., Tg, that is lower than the glass transition temperature of the ion-triggerable cationic polymer. Furthermore or alternatively, the co-binder polymer can be insoluble in water, or can reduce the shear viscosity of the ion-triggerable cationic polymer. The co-binder can be present at a level relative to the solids mass of the triggerable polymer of about 45 percent or less, specifically about 30 percent or less, more specifically about 20 percent or less, more specifically still about 15 percent or less, and most specifically about 10 percent or less, with exemplary ranges of from about 1 to about 45 percent or from about 25 to about 35 percent, as well as from about 1 to about 20 percent or from about 5 to about 25 percent. The amount of co-binder present should be low enough, for co-binders with the potential to form water insoluble bonds or films, that the co-binder remains a discontinuous phase unable to create enough crosslinked, or insoluble bonds, to jeopardize the dispersibility of the treated substrate.

Desirably, but not necessarily, the co-binder polymer when combined with the ion-triggerable cationic polymer will reduce the shear viscosity of the ion-triggerable cationic polymer to such an extent that the combination of the ion-triggerable cationic polymer and the co-binder polymer is sprayable. By sprayable is meant that the polymer can be applied to a nonwoven fibrous substrate by spraying and the distribution of the polymer across the substrate and the penetration of the polymer into the substrate are such that the polymer formulation is uniformly applied to the substrate.

In some embodiments, the combination of the ion-triggerable cationic polymer and the co-binder polymer can reduce the stiffness of the article to which it is applied compared to the article with just the ion-triggerable cationic polymer.

The co-binder polymer of the present disclosure can have an average molecular weight, which varies depending on the ultimate use of the polymer. Desirably, the co-binder polymer has a weight average molecular weight ranging from about 500,000 to about 200,000,000 grams per mol. More desirably, the co-binder polymer has a weight average molecular weight ranging from about 500,000 to about 100,000,000 grams per mol.

The co-binder polymer can be in the form of an emulsion latex. The surfactant system used in such a latex emulsion should be such that it does not substantially interfere with the dispersibility of the ion-triggerable cationic polymer. Therefore, weakly anionic, nonionic, or cationic latexes may be useful for the present disclosure. In one embodiment, the ion-triggerable cationic polymer formulations of the present disclosure comprises about 55 to about 95 weight percent ion-triggerable cationic polymer and about 5 to about 45 weight percent poly(ethylene-vinyl acetate). More desirably, the ion-triggerable cationic polymer formulations of the present disclosure comprises about 75 weight percent ion-triggerable cationic polymer and about 25 weight percent poly(ethylene-vinyl acetate). A particularly preferred non-crosslinking poly(ethylene-vinyl acetate) is Dur-O-Set® RB available from National Starch and Chemical Co., Bridgewater, N.J.

When a latex co-binder, or any potentially crosslinkable co-binder, is used the latex should be prevented from forming substantial water-insoluble bonds that bind the fibrous substrate together and interfere with the dispersibility of the article. Thus, the latex can be free of crosslinking agents, such as N-methylol-acrylamide (NMA), or free of catalyst for the crosslinker, or both. Alternatively, an inhibitor can be added that interferes with the crosslinker or with the catalyst such that crosslinking is impaired even when the article is heated to normal crosslinking temperatures. Such inhibitors can include free radical scavengers, methyl hydroquinone, t-butylcatechol, pH control agents such as potassium hydroxide, and the like. For some latex crosslinkers, such as N-methylol-acrylamide (NMA), for example, elevated pH such as a pH of 8 or higher can interfere with crosslinking at normal crosslinking temperatures (e.g., about 130° C. or higher). Also alternatively, an article comprising a latex co-binder can be maintained at temperatures below the temperature range at which crosslinking takes place, such that the presence of a crosslinker does not lead to crosslinking, or such that the degree of crosslinking remains sufficiently low that the dispersibility of the article is not jeopardized. Also alternatively, the amount of crosslinkable latex can be kept below a threshold level such that even with crosslinking, the article remains dispersible. For example, a small quantity of crosslinkable latex dispersed as discrete particles in an ion-sensitive binder can permit dispersibility even when fully crosslinked. For the later embodiment, the amount of latex can be below about 20 weight percent, and, more specifically, below about 15 weight percent relative to the ion-sensitive binder.

Latex compounds, whether crosslinkable or not, need not be the co-binder. SEM micrography of successful ion-sensitive binder films with useful non-crosslinking latex emulsions dispersed therein has shown that the latex co-binder particles can remain as discrete entities in the ion-sensitive binder, possibly serving in part as filler material. It is believed that other materials could serve a similar role, including a dispersed mineral or particulate filler in the triggerable binder, optionally comprising added surfactants/dispersants. For example, in one envisioned embodiment, free flowing Ganzpearl™ PS-8F particles from Presperse, Inc. (Piscataway, N.J.), a styrene/divinylbenzene copolymer with about 0.4 micron particles, can be dispersed in a triggerable binder at a level of about 2 to 10 weight percent to modify the mechanical, tactile, and optical properties of the triggerable binder. Other filler-like approaches may include microparticles, microspheres, or microbeads of metal, glass, carbon, mineral, quartz, and/or plastic, such as acrylic or phenolic, and hollow particles having inert gaseous atmospheres sealed within their interiors. Examples include EXPANCEL™ phenolic microspheres, which expand substantially when heated, or the acrylic microspheres known as PM™ 6545. Foaming agents, including CO₂ dissolved in the triggerable binder, could also provide helpful discontinuities as gas bubbles in the matrix of a triggerable binder, allowing the dispersed gas phase in the triggerable binder to serve as the co-binder. In general, any compatible material that is not miscible with the binder, especially one with adhesive or binding properties of its own, can be used as the co-binder, if it is not provided in a state that imparts substantial covalent bonds joining fibers in a way that interferes with the water-dispersibility of the product. However, those materials that also provide additional benefits, such as reduced spray viscosity, can be especially preferred. Adhesive co-binders, such as latex that do not contain crosslinkers or contain reduced amounts of crosslinkers, have been found to be especially helpful in providing good results over a wide range of processing conditions, including drying at elevated temperatures.

The co-binder polymer can comprise surface active compounds that improve the wettability of the substrate after application of the binder mixture. Wettability of a dry substrate that has been treated with a triggerable polymer formulation can be a problem in some embodiments, because the hydrophobic portions of the triggerable polymer formulation can become selectively oriented toward the air phase during drying, creating a hydrophobic surface that can be difficult to wet when the wetting composition is later applied unless surfactants are added to the wetting composition. Surfactants, or other surface active ingredients, in co-binder polymers can improve the wettability of the dried substrate that has been treated with a triggerable polymer formulation. Surfactants in the co-binder polymer should not significantly interfere with the triggerable polymer formulation. Thus, the binder should maintain good integrity and tactile properties in the pre-moistened wipes with the surfactant present.

In one embodiment, an effective co-binder polymer replaces a portion of the ion-triggerable cationic polymer formulation and permits a given strength level to be achieved in a pre-moistened wipe with at least one of lower stiffness, better tactile properties (e.g., lubricity or smoothness), or reduced cost, relative to an otherwise identical pre-moistened wipe lacking the co-binder polymer and comprising the ion-triggerable cationic polymer formulation at a level sufficient to achieve the given tensile strength.

The Dry Emulsion Powder (DEP) binders of Wacker Polymer Systems (Burghausen, Germany) such as the VINNEK™ system of binders, can be applied in some embodiments of the present disclosure. These are redispersible, free flowing binder powders formed from liquid emulsions. Small polymer particles from a dispersion are provided in a protective matrix of water soluble protective colloids in the form of a powder particle. The surface of the powder particle is protected against caking by platelets of mineral crystals. As a result, polymer particles that once were in a liquid dispersion are now available in a free flowing, dry powder form that can be redispersed in water or turned into swollen, tacky particles by the addition of moisture. These particles can be applied in high loft nonwovens by depositing them with the fibers during the airlaid process, and then later adding 10 to 30 percent moisture to cause the particles to swell and adhere to the fibers. This can be called the “chewing gum effect,” meaning that the dry, non-tacky fibers in the web become sticky like chewing gum once moistened. Good adhesion to polar surfaces and other surfaces is obtained. These binders are available as free flowing particles formed from latex emulsions that have been dried and treated with agents to prevent cohesion in the dry state. They can be entrained in air and deposited with fibers during the airlaid process, or can be applied to a substrate by electrostatic means, by direct contact, by gravity feed devices, and other means. They can be applied apart from the binder, either before or after the binder has been dried. Contact with moisture, either as liquid or steam, rehydrates the latex particles and causes them to swell and to adhere to the fibers. Drying and heating to elevated temperatures (e.g., above 160° C.) causes the binder particles to become crosslinked and water resistant, but drying at lower temperatures (e.g., at 110° C. or less) can result in film formation and a degree of fiber binding without seriously impairing the water dispersibility of the pre-moistened wipes. Thus, it is believed that the commercial product can be used without reducing the amount of crosslinker by controlling the curing of the co-binder polymer, such as limiting the time and temperature of drying to provide a degree of bonding without significant crosslinking.

As pointed out by Dr. Klaus Kohlhammer in “New Airlaid Binders,” Nonwovens Report International, September 1999, issue 342, pp. 20-22, 28-31, dry emulsion binder powders have the advantage that they can easily be incorporated into a nonwoven or airlaid web during formation of the web, as opposed to applying the material to an existing substrate, permitting increased control over placement of the co-binder polymer. Thus, a nonwoven or airlaid web can be prepared already having dry emulsion binders therein, followed by moistening when the ion-triggerable cationic polymer formulation solution is applied, whereupon the dry emulsion powder becomes tacky and contributes to binding of the substrate. Alternatively, the dry emulsion powder can be entrapped in the substrate by a filtration mechanism after the substrate has been treated with triggerable binder and dried, whereupon the dry emulsion powder is rendered tacky upon application of the wetting composition.

In another embodiment, the dry emulsion powder is dispersed into the triggerable polymer formulation solution either by application of the powder as the ion-triggerable cationic polymer formulation solution is being sprayed onto the web or by adding and dispersing the dry emulsion powder particles into the ion-triggerable cationic polymer formulation solution, after which the mixture is applied to a web by spraying, by foam application methods, or by other techniques known in the art.

In some embodiments of the present disclosure, the combination of an embossed layered substrate and the binder composition gives the dispersible wipe a geometric mean tensile (GMT) strength of at least about 300 grams per inch (g/n). In other embodiments of the present disclosure, the dispersible wipe has a GMT greater than about 250 g/n, and more preferably greater than about 275 Win and still more preferably greater than about 300 g/n, such as from about 250 to about 500 g/n, such as from about 250 to about 400 g/n.

In other embodiments of the present disclosure, the combination of an embossed layered substrate and the binder composition gives the dispersible wipe a GMT soak wet strength of less than about 180 g/n. In other embodiments of the present disclosure, the dispersible wipe has a GMT soak strength of less than about 175 g/n, less than about 170 g/n, less than about 165 g/n, less than about 160 g/n, less than about 155 g/n, less than about 150 g/n, less than about 145 g/n, or less than about 140 g/n. In some preferred embodiments of the present disclosure, the dispersible wipe has a GMT soak wet strength of from about 130 g/n to about 175 g/n.

In some preferred embodiments of the present disclosure, the combination of an embossed layered substrate and the binder composition gives the dispersible wipe a GMT strength of from about 250 g/n to about 400 g/n and a GMT soak wet strength of from about 130 g/n to about 175 g/n.

As noted elsewhere throughout this disclosure, forming a wipe substrate having two layers of differing density, embossing the substrate and treating the embossed substrate with a binder compositions creates a wipe with good dispersibility. The dispersibility of the dispersible wet wipes can be measured using a slosh-box test, as detailed elsewhere in this disclosure. In some embodiments of the present disclosure, the dispersible wipe of the present disclosure has a slosh-box break-up time of less than about 60 minutes, yet has sufficient strength to withstand use, such as a GMT strength of from about 250 to about 400 g/n. In other embodiments, the dispersible wipe has a slosh-box break-up time of from about 20 minutes to about 60 minutes, such as from about 30 to about 45 minutes. In some preferred embodiments of the present disclosure, the dispersible wipe has a GMT wet strength of at least about 250 g/n, a GMT soak wet strength of less than about 180 g/n and a slosh-box break-up time of less than about 60 minutes. In other embodiments of the present disclosure, the dispersible wipe has a GMT strength of from about 250 to about 400 g/n, a GMT soak wet strength of from about 130 g/n to about 175 g/n and a slosh-box break-up time of from about 30 minutes to about 60 minutes.

In other embodiments the disclosure provides an embossed dispersible wipe having two layers of differing density and ion triggerable binder where the wipe has a GMT from about 250 to about 400 Win., a burst strength greater than about 300 gf, more preferable greater than about 315 gf and still more preferable greater than about 330 gf, such as from about 300 to about 350 gf. Thus, wipes prepared according to the present disclosure may have sufficient strength to withstand use, but have good durability. Further, the foregoing wipes may be readily dispersed, such as having a slosh-box break-up time of from about 20 minutes to about 60 minutes, such as from about 30 to about 45 minutes.

The strength of the dispersible nonwoven sheets generated from each example can be evaluated by measuring the tensile strength in the machine direction and the cross-machine direction. Tensile strength can be measured using a Constant Rate of Elongation (CRE) tensile tester having a 1-inch jaw width (sample width), a test span of 3 inches (gauge length), and a rate of jaw separation of 25.4 centimeters per minute after soaking the sheet in tap water for 4 minutes and then draining the sheet on dry Viva® brand paper towel for 20 seconds. This drainage procedure can result in a moisture content of 200 percent of the dry weight +/−50 percent. This can be verified by weighing the sample before each test. One-inch wide strips can be cut from the center of the dispersible nonwoven sheets 80 in the specified machine direction 24 (“MD”) or cross-machine direction 25 (“CD”) orientation using a JDC Precision Sample Cutter (Thwing-Albert Instrument Company, Philadelphia, Pa., Model No. JDC3-10, Ser. No. 37333). The “MD tensile strength” is the peak load in grams-force per inch of sample width when a sample is pulled to rupture in the machine direction. The “CD tensile strength” is the peak load in grams-force per inch of sample width when a sample is pulled to rupture in the cross direction.

The instrument used for measuring tensile strength can be an MTS Systems Synergie 200 model and the data acquisition software can be MTS TestWorks® for Windows Ver. 4.0 commercially available from MTS Systems Corp., Eden Prairie, Minn. The load cell can be an MTS 50 Newton maximum load cell. The gauge length between jaws can be 3±0.04 inches and the top and bottom jaws can be operated using pneumatic-action with maximum 60 P.S.I. The break sensitivity can be set at 70 percent. The data acquisition rate can be set at 100 Hz (i.e., 100 samples per second). The sample can be placed in the jaws of the instrument, centered both vertically and horizontally. The test can be then started and ended when the force drops by 70 percent of peak. The peak load can be expressed in grams-force and can be recorded as the “MD tensile strength” of the specimen. All of these values are for in-use tensile strength measurements.

The Soak Wet Strength was carried out by soaking the 1″ wide strips described above for the tensile testing in a bath of 4.1 liter of deionized water for 1 hour. The deionized water was not stirred or agitated in any way during the testing. At the completion of the 1 hour soak, each of the samples were carefully retrieved from the bath, allowed to drain to remove excess water, and then tested immediately as described above for the tensile testing.

The Slosh-Box Test uses a bench-scaled apparatus to evaluate the breakup or dispersibility of flushable consumer products as they travel through the wastewater collection system. In this test, a clear plastic tank is loaded with a product and tap water or raw wastewater. The container is then moved up and down by a cam system at a specified rotational speed to simulate the movement of wastewater in the collection system. The initial breakup point and the time for dispersion of the product into pieces measuring 1 inch by 1 inch (25 mm by 25 mm) are recorded in the laboratory notebook. This 1 inch by 1 inch (25 mm by 25 mm) size is a parameter that is used because it reduces the potential of product recognition. The various components of the product can then be screened and weighed to determine the rate and level of disintegration.

The slosh-box water transport simulator may consist of a transparent plastic tank that can be mounted on an oscillating platform with speed and holding time controller. The angle of incline produced by the cam system produces a water motion equivalent to 60 cm/s (2 ft/s), which is the minimum design standard for wastewater flow rate in an enclosed collection system. The rate of oscillation was controlled mechanically by the rotation of a cam and level system and was measured periodically throughout the test. This cycle mimics the normal back-and forth movement of wastewater as it flows through sewer pipe.

Room temperature tap water can be placed in the plastic container/tank. The timer can be set for six hours (or longer) and cycle speed can be set for 26 rpm. The pre-weighed product can be placed in the tank and observed as it undergoes the agitation period. The time to first breakup and full dispersion can be recorded in the laboratory notebook.

The test can be terminated when the product reaches a dispersion point of no piece larger than 1 inch by 1 inch (25 mm by 25 mm) square in size. At this point, the clear plastic tank can be removed from the oscillating platform. The entire contents of the plastic tank can then be poured through a nest of screens arranged from top to bottom in the following order: 25.40 mm, 12.70 mm, 6.35 mm, 3.18 mm, 1.59 mm (diameter opening). With a showerhead spray nozzle held approximately 10 to 15 cm (4 to 6 in) above the sieve, the material can be gently rinsed through the nested screens for two minutes at a flow rate of 4 L/min (1 gal/min) being careful not to force passage of the retained material through the next smaller screen. After two minutes of rinsing, the top screen can be removed and the rinsing can be continued for the next smaller screen, still nested, for two additional minutes. After rinsing, the retained material can be removed from each of the screens using forceps. The contents can be transferred from each screen to a separate, labeled aluminum weigh pan. The pan can be placed in a drying oven overnight at 103 ±3° C. The dried samples can be allowed to cool down in a desiccator. After all the samples are dry, the materials from each of the retained fractions can be weighed and the percentage of disintegration based on the initial starting weight of the test material can be calculated.

Burst strength may be measured substantially as described in U.S. Pat. No. 5,667,635 using a tensile tester equipped with a computerized data-acquisition system that is capable of calculating peak load and energy between two predetermined distances (15-60 millimeters). The load cell should be chosen so that the peak load values fall between 10 and 90 percent of the full-scale load for the material being tested. Suitable tensile testers include the MTS Systems Synergie 200 model and the data acquisition software can be MTS TestWorks® for Windows Ver. 4.0 commercially available from MTS Systems Corp., Eden Prairie, Minn.

The burst test is carried out in a standard laboratory atmosphere of about 23° C. and about 50 percent relative humidity. The test instrument should be mounted on a table free of vibrations to avoid ending the test prematurely. The sample, soaked in tap water for 4 minutes and then draining the sheet on dry Viva® brand paper towel for 20 seconds, is draped across the opening of the sample stand and secured with the magnetic ring. The inside diameter of the sample stand is 2.5 inches and the inside diameter of the magnetic ring is 2.82 inches. The probe is aluminum and has a length of 4.5 inches, a diameter of 0.50 inch and a radius of curvature at the end of 0.25 inch. During the test, the probe is lowered onto the sample at a rate of 16 inches per minute until the sample tears. The peak load is the wet burst strength for the sample, having units of grams force (gf). A representative number of samples should be tested to obtain an average value, which is the burst strength.

### EXAMPLES

Basesheets were made using a through-air dried papermaking process commonly referred to as “uncreped through-air dried” (“UCTAD”) and generally described in U.S. Pat. No. 5,607,551, the contents of which are incorporated herein in a manner consistent with the present disclosure. The basesheets had a target bone dry basis weight of about 45 grams per square meter (gsm). In all cases the basesheets were produced from a furnish consisting entirely of northern softwood kraft pulp.

The tissue web was formed on a Voith Fabrics TissueForm V forming fabric, vacuum dewatered to approximately 25 percent consistency and then subjected to rush transfer when transferred to the transfer fabric. The transfer fabric was the fabric described as “Fred” in U.S. Pat. No. 7,611,607 (commercially available from Voith Fabrics, Appleton, Wis.). The web was then transferred to a through-air drying fabric comprising a printed silicone pattern disposed on the sheet contacting side. The silicone formed a wave-like pattern on the sheet contacting side of the fabric. The control was prepared using the through-air drying fabric described as “Fozzie” in US Publication No. 2015/0247290 A1. The pattern properties of the control and inventive fabrics are summarized in Table 1, below.

TABLE 1

| Element | Element CD | Pattern | Pattern |
|---|---|---|---|
| Height (mm) | Spacing (mm) | Wavelength (mm) | Amplitude (mm) |
| 0.9 | 4.1 | 100 | 20 |

Transfer to the through-drying fabric was done using vacuum levels of about 10 inches of mercury at the transfer. The web was then dried to approximately 98 percent solids before winding.

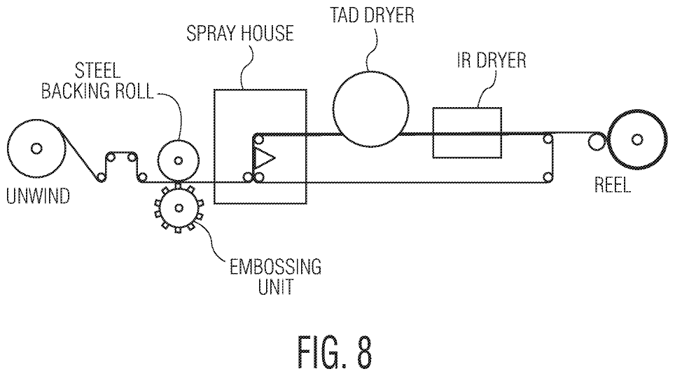

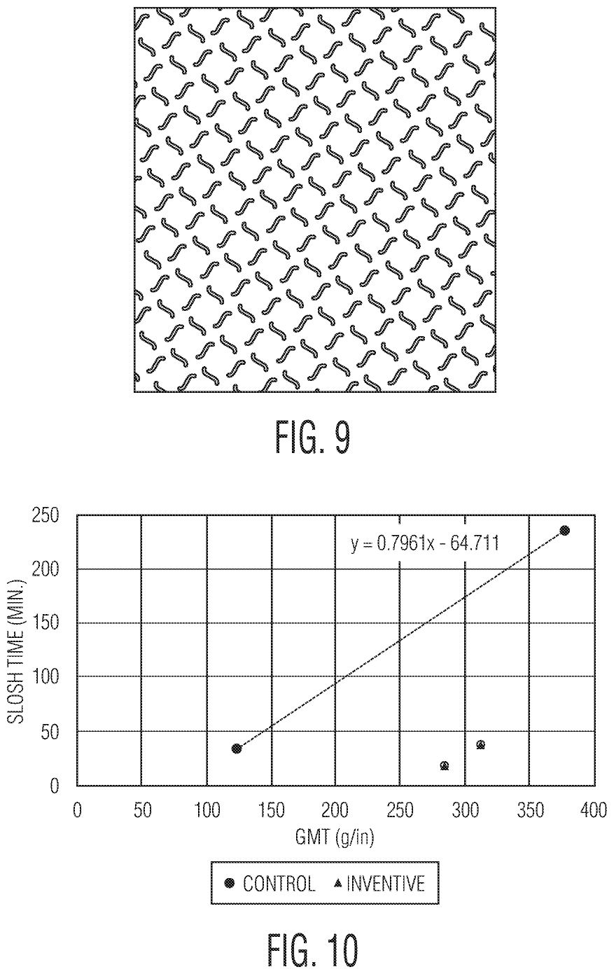

Basesheet was converted into a dispersible wipe substantially as illustrated in FIG. 8. Basesheet was unwound and embossed by passing the basesheet through an embossing nip created by a patterned steel embossing roll and steel backing roll. The embossing nip pressure was approximately 122 pli. Two different embossing patterns were evaluated, as indicated in Table 2, below. The embossed web was transferred to an oven wire, where it was sprayed on the top side with a series of Unijet® nozzles, Nozzle type 800050 (Spraying Systems Co., Wheaton, Ill.), operating at approximately 80 psi. The binder composition was sprayed at approximately 15 percent binder solids with water as the carrier. The binder composition comprised a cationic polyacrylate resulting from the polymerization of 96 mol percent methyl acrylate and 4 mol percent [2-(acryloyloxy)ethyl]trimethyl ammonium chloride and a co-binder, VINNAPAS® EZ123. The ratio of binder to co-binder was 70:30. The total add-on binder was 4.0 grams per square meter of web.

TABLE 2

| Sample | Embossing Pattern |
|---|---|
| Control 1 | NA |
| Control 2 | NA |
| Inventive 1 | FIG. 6 |
| Inventive 2 | FIG. 8 |

After spraying with the binder the web was dried through a series of dryers comprising a through-air dryer and an infra-red dryer operating from about 220 to 260° C. at a speed of about 200 feet per minute (fpm) to dry the web. The dried web was then passed through the same series of driers a second time at a speed of about 100 to about 200 fpm to cure the binder.

The cured and dried web was then converted into sections of continuous web separated by rows of perforations and wetted with a wetting composition at 235 percent add-on to yield a fan-folded stack of wet wipes. The wetting composition was substantially similar that used on commercially available wet wipes under the trade designation KLEENEX® COTTON ELLE FRESH® Folded Wipes (Kimberly-Clark Corporation of Neenah, Wis.).

The exemplary dispersible wipes were subjected to physical testing, the results of which are summarized in Tables 3 and 4, below. To determine the improvement in dispersability for the inventive samples a time to break-up (Slosh Time) curve was constructed from the control samples based upon their measured geometric mean tensile strengths and Slosh Times. The curve is shown in FIG. 10. Estimated Slosh Times were calculated based the control curve (FIG. 10) and the geometric mean tensile strengths of the inventive samples. In each instance the inventive samples had significantly reduced Slosh Times compared to their estimated Slosh Times.

TABLE 3

| | Basis | | MD | MD TEA | MD | CD | CD TEA | CD | Slosh |
|---|---|---|---|---|---|---|---|---|---|
| | Wt. | Caliper | Tensile | (gf*cm/ | Stretch | Tensile | (gf*cm/ | Stretch | Time |
| Sample | (gsm) | (mm) | (gf) | cm\hat{ }2) | (%) | (gf) | cm\hat{ }2) | (%) | (min.) |
| Control 1 | 46 | 0.38 | 670 | 17.50 | 12.00 | 212.7 | 11.5 | 17.7 | 124 |
| Control 2 | 40 | 0.39 | 434 | 13.30 | 13.20 | 128.1 | 7.5 | 13.8 | 34 |
| Inventive 1 | 46 | 0.33 | 584 | 14.50 | 11.40 | 167.0 | 9.1 | 15.9 | 38 |
| Inventive 2 | 45 | 0.30 | 491 | 12.90 | 10.80 | 164.6 | 10.2 | 17.3 | 18 |

TABLE 4

| | | GM | GM TEA | Burst | Estimated | Slosh Time |
|---|---|---|---|---|---|---|
| | GMT | Stretch | (gf*cm/ | Peak Load | Slosh Time | Improvement |
| Sample | (gf) | (%) | cm\hat{ }2) | (gf) | (min.) | (%) |
| Control 1 | 377.5 | 14.6 | 14.19 | 428 | NA | NA |
| Control 2 | 235.8 | 13.5 | 9.99 | 177 | NA | NA |
| Inventive 1 | 312.3 | 13.5 | 11.49 | 318 | 184 | 79% |
| Inventive 2 | 284.3 | 13.7 | 11.47 | 311 | 162 | 89% |

### EMBODIMENTS

First embodiment: A dispersible wet wipe comprising: a wipe substrate having at least a first outer layer comprising a tissue web containing cellulose fibers and a plurality of embossments disposed thereon, and a second outer layer comprising a nonwoven web; a triggerable binder composition; and a wetting composition, wherein the dispersible wet wipe has a geometric mean tensile strength (GMT) greater than about 225 grams per linear inch (g/in) and a Slosh Time less than about 60 minutes.

Second embodiment: The wipe of the first embodiment wherein the triggerable binder composition is present at an add-on rate of between about 1 and about 8 percent based on the total weight of the wipe substrate.

Third embodiment: The wipe of any one of embodiments 1 or 2 wherein the tissue web comprises an uncreped through-air dried tissue web.

Fourth embodiment: The wipe of any one of embodiments 1 through 3 wherein the first outer layer has a density of between about 0.5 and 2.0 grams per cubic centimeter and the second outer layer has a density of between about 0.05 and 0.15 grams per cubic centimeter.

Fifth embodiment: The wipe of any one of embodiments 1 through 4 wherein the triggerable binder composition is ion-triggerable.

Sixth embodiment: The wipe of any one of embodiments 1 through 5 wherein the plurality of embossments are disposed in a pattern and the total embossed area ranges from about 5 to about 15 percent.

Seventh embodiment: The wipe of any one of embodiments 1 through 6 wherein the plurality of embossments consist essentially of curvilinear line elements having a line width from about 0.5 to about 2.5 mm.

Eighth embodiment: The wipe of any one of embodiments 1 through 7 wherein the plurality of embossments are disposed in a pattern having an axis of orientation and the axis of orientation is substantially aligned in the cross-machine or machine direction.

Ninth embodiment: The wipe of any one of embodiments 1 through 8 wherein the plurality of embossments are discrete and wherein embossments are disposed in a pattern consisting of substantially similarly shaped motifs each having a pillow region having a maximum dimension of less than 2.5 cm.

Tenth embodiment: The wipe of any one of embodiments 1 through 9 wherein the wipe has a GMT strength of from about 250 to about 400 g/n, a GMT soak wet strength from about 130 g/n to about 175 g/n and a slosh-box break-up time from about 30 minutes to about 60 minutes.

Eleventh embodiment: The wipe of any one of embodiments 1 through 10 wherein the wipe has a GMT from about 250 to about 400 g/n and a burst strength greater than about 300 gf.

Twelfth Embodiment: The wipe of any one of embodiments 1 through 11 wherein the wipe wherein the tissue web further comprises a background pattern.

Thirteenth Embodiment: The wipe of any one of embodiments 1 through 12 wherein the wipe wherein the tissue web further comprises a background pattern consisting essentially of a plurality of continuous, spaced apart, parallel, line elements.

What is claimed is:

1. A dispersible wet wipe comprising: a wipe substrate having at least a first outer layer comprising a tissue web containing cellulose fibers and a plurality of embossments disposed thereon, and a second outer layer comprising a nonwoven web; a triggerable binder composition; and a wetting composition, wherein the dispersible wet wipe has a geometric mean tensile strength (GMT) greater than about 250 grams per linear inch (g/in) and a Slosh Time less than about 60 minutes.

2. The dispersible wet wipe of claim 1 having a GMT from about 250 to about 400 g/n and a burst strength greater than about 300 grams force (gf).

3. The dispersible wet wipe of claim 1 wherein the triggerable binder composition is present at an add-on rate of between about 1 and about 8 percent based on the total weight of the wipe substrate.

4. The dispersible wet wipe of claim 1 wherein the tissue web comprises an uncreped through-air dried tissue web.

5. The dispersible wet wipe of claim 1 wherein the first outer layer has a density of between about 0.5 and 2.0 grams per cubic centimeter and the second outer layer has a density of between about 0.05 and 0.15 grams per cubic centimeter.

6. The dispersible wet wipe of claim 1 wherein the triggerable binder composition is ion-triggerable.

7. The dispersible wet wipe of claim 1 wherein the triggerable binder composition has the structure: 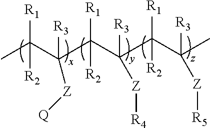
wherein x=1 to about 15 mole percent; y=about 60 to about 99 mole percent; and z=0 to about 30 mole percent; Q is selected from C₁-C₄alkyl ammonium, quaternary C₁-C₄ alkyl ammonium and benzyl ammonium; Z is selected from —O—, —OOO—, —OOC—, —CONH—, and —NHCO—; R₁, R₂, R₃ are independently selected from hydrogen and methyl; R₄ is C₁-C₄ alkyl; R₅ is selected from hydrogen, methyl, ethyl, butyl, ethylhexyl, decyl, dodecyl, hydroxyethyl, hydroxypropyl, polyoxyethylene, and polyoxypropylene.

8. The dispersible wet wipe of claim 1 wherein the plurality of embossments are disposed in a pattern and the total embossed area ranges from about 5 to about 15 percent and the plurality of embossments consist essentially of curvilinear line elements having a line width from about 0.5 to about 2.5 mm.

9. The dispersible wet wipe of claim 1 wherein the plurality of embossments are disposed in a pattern having an axis of orientation and the axis of orientation is substantially aligned in the cross-machine or machine direction.

10. The dispersible wet wipe of claim 1 wherein the plurality of embossments consist essentially of discrete embossed line elements having a width from about 0.5 to about 2.5 mm.

11. The dispersible wet wipe of claim 10 wherein the discrete embossed line elements are curvilinear and the total embossed area ranges from about 5 to about 15 percent.

12. A method of forming a dispersible wet wipe comprising the steps of:
a) forming a first fibrous layer comprising a cellulosic tissue web;
b) forming a second fibrous layer comprising a nonwoven web;
c) combining the first and the second layers to form a multi-layered web;
d) conveying the multi-layered web through an embossing nip;
e) embossing the multi-layered web thereby forming a plurality of embossments on at least the first fibrous layer of the multi-layered web; and
f) applying a triggerable binder composition to at least the first or the second fibrous layer of the multi-layered web.

13. The method of claim 12 further comprising the step of curing the multi-layered web.

14. The method of claim 12 further comprising the step of wetting the multi-layered web and wherein the wetting step is done before conveying the multi-layered web through an embossing nip.

15. The method of claim 12 wherein the triggerable binder composition is applied at an add-on rate of between about 1 and about 8 percent based on the total weight of the multi-layered fibrous web.

16. The method of claim 12 wherein the tissue web comprises an uncreped through-air dried tissue web having a density of between about 0.5 and 2.0 grams per cubic centimeter and the second outer layer has a density of between about 0.05 and 0.15 grams per cubic centimeter.

17. The method of claim 12 wherein the triggerable binder composition is ion-triggerable.

18. The method of claim 12 wherein the plurality of embossments are disposed in a pattern and the total embossed area ranges from about 5 to about 15 percent.

19. The method of claim 12 wherein the plurality of embossments consist essentially of curvilinear line elements having a line width from about 0.5 to about 2.5 mm.

20. The method of claim 12 wherein the plurality of embossments consist essentially of discrete embossed elements.
